(19) United States
(12) Patent Application Publication
(10) Pub. No.: US 2024/0211236 A1
(43) Pub. Date: Jun. 27, 2024

(54) SYSTEMS AND METHODS FOR UPGRADING MEDICAL DEVICES

(71) Applicant: WUHAN UNITED IMAGING HEALTHCARE CO., LTD., Wuhan (CN)

(72) Inventor: Chao YUAN, Wuhan (CN)

(73) Assignee: WUHAN UNITED IMAGING HEALTHCARE CO., LTD., Wuhan (CN)

(21) Appl. No.: 18/389,708

(22) Filed: Dec. 19, 2023

(30) Foreign Application Priority Data
Dec. 22, 2022 (CN) 202211651721.X

(51) Int. Cl. G06F 8/65; G06F 8/71; G16H 40/40

(52) U.S. Cl. CPC G06F 8/65; G06F 8/71; G16H 40/40

(57) ABSTRACT

The present disclosure provides a system and method for upgrading medical devices, comprising: a registration center, at least one upgrade server and at least one medical device, wherein the registration center is connected to the at least one upgrade server, and a plurality of upgrade service modules are deployed on the at least one upgrade server, each of the at least one upgrade service module being deployed on one of the at least one upgrade server.

### CROSS REFERENCE TO RELATED APPLICATION

This application claims priority to Chinese application No. 202211651721. X, filed on Dec. 22, 2022, the entire contents of which are incorporated herein by reference.

### TECHNICAL FIELD

The present disclosure relates to the medical field, and in particular, to systems and methods for upgrading medical devices.

### BACKGROUND

To ensure data security, a medical device is usually connected to a local area network (LAN), which limits the way in which the medical device is upgraded and maintained. For example, a traditional way of upgrading the medical device requires a user to manually upgrade the medical device at a site where the medical device is located, or connect the medical device to a public network to receive an upgrade package from a remote maintenance platform. However, since there is a plurality of medical devices in each hospital, when a count of medical devices that need to be upgraded is high, the above method requires a lot of human and material resources, and the upgrade efficiency is low.

### SUMMARY

Embodiments of the present disclosure provide a system for upgrading medical devices, comprising a registration center and at least one upgrade server, and at least one medical device, the registration center being connected to the at least one upgrade server and the at least one medical device, and at least one upgrade service module being deployed on the at least one upgrade server, each of the at least one upgrade service module being deployed on one of the at least one upgrade server, wherein the registration center is configured for, in response to an upgrade request sent by a target medical device, determining a target upgrade service module from the at least one upgrade service module and sending registration information of the target upgrade service module to the target medical device; the target medical device is configured for establishing a communication connection with the target upgrade service module based on the registration information and sending device information to the target upgrade service module through the communication connection; and the target upgrade service module is configured for determining a target upgrade package matching the target medical device based on the device information and sending the target upgrade package to the target medical device.

In some embodiments, wherein the determining a target upgrade service module from the at least one upgrade service module comprises determining the target upgrade service module from the at least one upgrade service module based on first load information of each upgrade service module.

In some embodiments, wherein the determining the target upgrade service module from the at least one upgrade service module based on first load information of each upgrade service module comprises: for each upgrade service module, obtaining the first load information of the upgrade service module and second load information of an upgrade server on which the upgrade service module is deployed; and determining the target upgrade service module from the at least one upgrade service module based on the first load information and the second load information corresponding to each upgrade service module.

In some embodiments, wherein when the registration center receives upgrade requests from a plurality of target medical devices at the same time, the registration center is further configured for determining a priority ranking of the plurality of target medical devices based on device information of the plurality of target medical devices; and determining a corresponding target upgrade service module for each of the plurality of target medical devices in turn based on the priority ranking of the plurality of target medical devices.

In some embodiments, wherein before receiving the upgrade request sent by the target medical device, the registration center is further configured for receiving registration information sent by the at least one upgrade service module and store the registration information.

In some embodiments, wherein one of the at least one upgrade service module is configured for sending a wake-up message to the target medical device based on a preset wake-up time; and the target medical device is further configured for sending the upgrade request to the registration center in response to the wake-up message.

In some embodiments, wherein when the target medical device is in a shutdown state or a standby state at a time of receiving the wake-up message, the sending the upgrade request to the registration center in response to the wake-up message comprises powering up and sending the upgrade request to the registration center; or when the target medical device is in a working state at the time of receiving the wake-up message, the sending the upgrade request to the registration center in response to the wake-up message comprises sending the upgrade request to the registration center after the target medical device has finished a current workflow.

In some embodiments, wherein the device information comprises version information of the target medical device, and the determining a target upgrade package matching the target medical device based on the device information received further comprises determining a current version of the target medical device based on the version information; in response to determining that the current version is lower than a target version corresponding to the target upgrade service module, determining an upgrade link for upgrading to the target version from the current version; and determining, based on the upgrade link, the target upgrade package matching the target medical device from at least one upgrade package of the target upgrade service module.

In some embodiments, wherein the at least one upgrade package of the target upgrade service module is obtained from a remote management device through a gateway.

In some embodiments, wherein the target upgrade service module is further configured for obtaining an upgrade result of the target medical device; and sending the upgrade result to a remote management device through a gateway.

In some embodiments, wherein the registration center is further configured for: for each upgrade service module, sending a heartbeat signal to the upgrade service module; determining a connection state between the upgrade service module and the registration center based on a response of the upgrade service module to the heartbeat signal; and in response to determining that the connection state is a disconnected state, deleting the registration information of the upgrade service module.

In some embodiments, wherein the registration center and the at least one upgrade server are deployed on a same server.

Embodiments of the present disclosure provide a method for device upgrading, wherein the method is implemented by a target medical device, comprising sending an upgrade request to a registration center such that the registration center determines a target upgrade service module from at least one upgrade service module based on the upgrade request; receiving registration information of the target upgrade service module sent by the registration center; establishing a communication connection with the target upgrade service module based on the registration information and sending device information to the target upgrade service module through the communication connection such that the target upgrade service module determines a target upgrade package based on the device information; and receiving the target upgrade package sent by the target upgrade service module and performing device upgrading based on the target upgrade package.

Embodiments of the present disclosure provide a method for device upgrading, implemented on a target upgrade service module deployed on an upgrade server, the upgrade server being connected to a registration center, wherein the method comprises establishing a communication connection to a target medical device, wherein the target upgrade service module is determined from at least one upgrade service module deployed on the upgrade server by the registration center in response to an upgrade request sent by a target medical device; receiving device information from the target medical device through the communication connection; determining a target upgrade package matching the target medical device based on the device information; and sending the target upgrade package to the target medical device.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary device upgrading system according to some embodiments of the present disclosure;

FIG. 2 is a schematic diagram illustrating another exemplary device upgrading system according to other embodiments of the present disclosure;

FIG. 3 is a schematic diagram illustrating exemplary software/hardware of a computing device according to some embodiments of the present disclosure.

FIG. 4 is a schematic diagram illustrating an exemplary device upgrading system according to some embodiments of the present disclosure;

FIG. 5 is a schematic diagram illustrating an exemplary process for reporting device information of a target medical device according to some embodiments of the present disclosure;

FIG. 6 is a schematic diagram illustrating an exemplary wake-up process of a medical device according to some embodiments of the present disclosure;

FIG. 7 is a schematic diagram illustrating exemplary device information stored in an upgrade service module according to some embodiments of the present disclosure;

FIG. 8 is a schematic diagram illustrating an exemplary process for determining a target upgrade package according to some embodiments of the present disclosure;

FIG. 9 is a schematic diagram illustrating an exemplary process of issuing an upgrade package from a remote management device according to some embodiments of the present disclosure;

FIG. 10 is a schematic diagram illustrating an exemplary process for upgrading a target medical device according to some embodiments of the present disclosure;

FIG. 11 is a schematic diagram illustrating an exemplary process for upgrading a target medical device according to some embodiments of the present disclosure;

FIG. 12 is a flowchart illustrating an exemplary process for upgrading a target medical device according to some embodiments of the present disclosure; and

FIG. 13 is a flowchart illustrating an exemplary process for upgrading a target medical device according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system”, “device”, “unit” and/or “module” used herein are a way to distinguish between different components, elements, parts, sections, or assemblies at different levels. However, the terms may be replaced by other expressions if other words accomplish the same purpose.

Usually, the terms “module”, “unit”, or “block” are used here to refer to the logic or collection of software instructions embodied in hardware or firmware, or a collection of software instructions. Modules, units, or blocks described herein may be implemented as software and/or hardware, and may be stored in any type of non-transitory computer-readable medium or another storage device. In some embodiments, the software module/unit/block may be compiled and linked into an executable program. It should be appreciated that the software modules may be called from other modules/units/blocks or from themselves, and/or may be called in response to a detected event or interrupt. Software modules/units/blocks configured to be executed on a computing device may be provided on a computer-readable medium (e.g., a CD-ROM, a digital video diskette, a flash drive, a diskette, or any other tangible medium), or as a digital download (which may initially be stored in a compressed or installable format that needs to be installed, decompressed, or decrypted prior to execution). Software code herein may be stored in part or in whole in a storage device of a computing device that performs an operation and is applied in the operation of the computing device. The software instructions may be embedded in firmware, such as an EPROM. It should also be understood that hardware modules/units/blocks may be included in connected logic assemblies, such as gates and flip-flops, and/or may include programmable units, such as programmable gate arrays or processors. The modules/units/blocks or computing device functions described herein may be implemented as software modules/units/blocks, but may be represented in hardware or firmware. Usually, the modules/units/blocks described herein refer to logical modules/units/blocks, which may be combined with other modules/units/blocks or divided into sub-modules/sub-units/sub-blocks, even though they are physical organizations or memory pieces. This description may apply to a system, an engine, or a portion thereof.

It is to be understood that, unless the context explicitly states otherwise, when a unit, engine, module, or block is said to be “on” another unit, engine, module, or block, “connected” or “coupled to” another unit, engine, module, or block, it may be directly on, connected or coupled to, or in communication with, other units, engines, modules, or blocks, or there may be intermediate units, engines, modules, or blocks. In the present disclosure, the term “and/or” may include any one or more of the relevant listed entries or combinations thereof. In the present disclosure, the term “image” may refer to a 2D image, a 3D image, or a 4D image.

These and other features, characteristics, and methods of functioning and operation of the relevant structural elements, as well as combinations of components and economies of manufacture of the present disclosure may be rendered more apparent in the light of the following description in conjunction with the accompanying drawings, which form a part. It should be understood, however, that the accompanying drawings are for illustrative and descriptive purposes only and are not intended to limit the scope of the present disclosure. It should be understood that the accompanying drawings are not to scale.

As shown in the present disclosure and in the claims, unless the context clearly suggests an exception, the words “one”, “a”, “an”, “one kind”, and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

Flowcharts are used in the present disclosure to illustrate the operations performed by a system according to embodiments of the present disclosure, and the related descriptions are provided to aid in a better understanding of the magnetic resonance imaging method and/or system. It should be appreciated that the preceding or following operations are not necessarily performed in an exact sequence. Instead, steps can be processed in reverse order or simultaneously. Also, it is possible to add other operations to these processes or to remove a step or steps from these processes.

As aforementioned, a traditional way of upgrading a medical device requires a user to manually upgrade the medical device after arriving at a site where the medical device is located, connect the medical device to a public network, and upgrade the medical device through an upgrade package issued by a remote maintenance platform. When the count of medical devices that need to be upgraded is large, the above method requires a lot of labor and material resources, and the upgrade efficiency is low.

Some embodiments of the present disclosure provide a system and method for upgrading medical devices. The system may include a registration center, at least one upgrade server, and at least one medical device, wherein the registration center is connected to the at least one upgrade server and the at least one medical device, and one or more upgrade service modules are deployed on each upgrade server of the at least one upgrade server. In response to an upgrade request sent by a target medical device, the registration center determines a target upgrade service module from the one or more upgrade service modules, and sends registration information of the target upgrade service module to the target medical device. The target medical device establishes a communication connection with the target upgrade service module based on the registration information, and reports device information to the target upgrade service module via the communication connection. The target upgrade service module determines a target upgrade package matching the target medical device based on the device information. The target upgrade service module issues the target upgrade package to the target medical device so that the target medical device can be upgraded.

The above method enables the upgrade service module(s) to simultaneously send upgrade data (e.g., target upgrade packages) to a plurality of medical devices (e.g., multiple target medical devices) to improve the efficiency of upgrading medical devices. Furthermore, the method may utilize a microservices framework (e.g., a registration center, upgrade service modules, a gateway, etc.) to realize concurrent processing of upgrade tasks by the upgrade service modules, so that the upgrade service modules may simultaneously issue upgrade data to a plurality of medical devices, thereby improving the efficiency of upgrading medical devices.

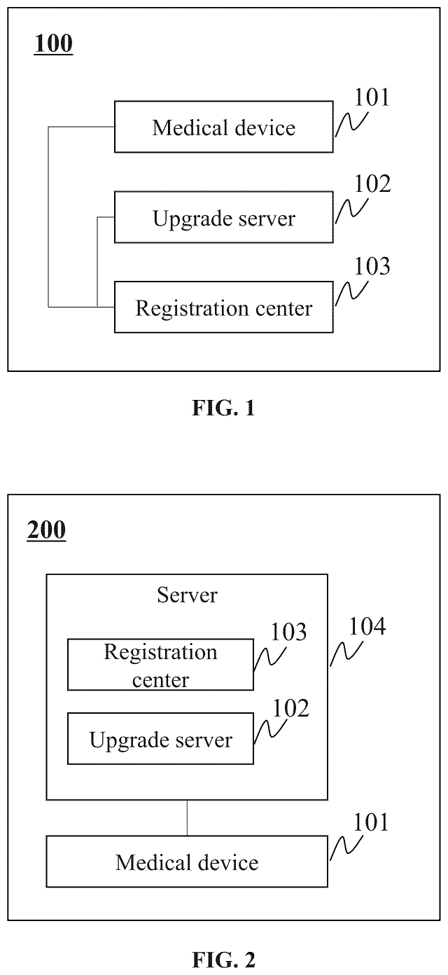

FIG. 1 is a schematic diagram illustrating an exemplary device upgrading system 100 according to some embodiments of the present disclosure.

As shown in FIG. 1, the device upgrading system 100 may include a medical device 101, an upgrade server 102, and a registration center 103. Components of the device upgrading system 100 may be interconnected to each other. For example, as shown in FIG. 1, the registration center 103 may be connected to the upgrade server 102 and the medical device 101, respectively.

The medical device 101 may be a device that needs to receive an upgrade service. The medical device 101 may include but is not limited to, an imaging device, a therapeutic device, and an assistive device. For example, the medical device 101 may be an ultrasound device, a magnetic resonance device, an X-ray device, a computed tomography (CT) device, a fluorescence fluoroscopy imaging component, a single-photon emission computed tomography (SPECT) device, positron emission tomography (PET) device, radiation therapy device, and a combination of one or more of the same. A count of the medical device 101 may be one or more.

One or more upgrade service modules may be deployed on the upgrade server 102. An upgrade service module may refer to software (e.g., a computer program) deployed on a computer and configured to provide upgrade services for the medical device 101 (e.g., issuing a target upgrade package to the medical device 101). A count of the upgrade server 102 may be one or more. Each upgrade service module may be deployed on one upgrade server. The one or more upgrade service modules may be deployed on a single upgrade server 102, or may be deployed on different upgrade servers. For example, an upgrade service module 1 may be deployed on an upgrade server 1, an upgrade service module 2 may be deployed on an upgrade server 2, an upgrade service module 3 may be deployed on an upgrade server 3. As another example, the upgrade service module 1, the upgrade service module 2, the upgrade service module 3 may be deployed on the upgrade server 1, and an upgrade service module 4 and an upgrade service module 5 may be deployed on the upgrade server 2.

The registration center 103 may serve as a manager of the device upgrading system 100. For example, the registration center 103 may receive and deal with an upgrade request sent by a target medical device and assign a target upgrade service module to the target medical device. As another example, the registration center 103 may receive registration information sent by a plurality of upgrade service modules and store the registration information.

In some embodiments, the registration center 103 may be deployed on a registration server. A count of the registration center 103 may be one or more. When there is a plurality of registration center 103, the plurality of registration center 103 may be deployed on a single registration server (e.g., in the form of a plurality of registration software), or may be deployed on a plurality of registration servers. For example, a registration center 1 may be deployed on a registration server 1, and a registration center 2 may be deployed on a registration server 2.

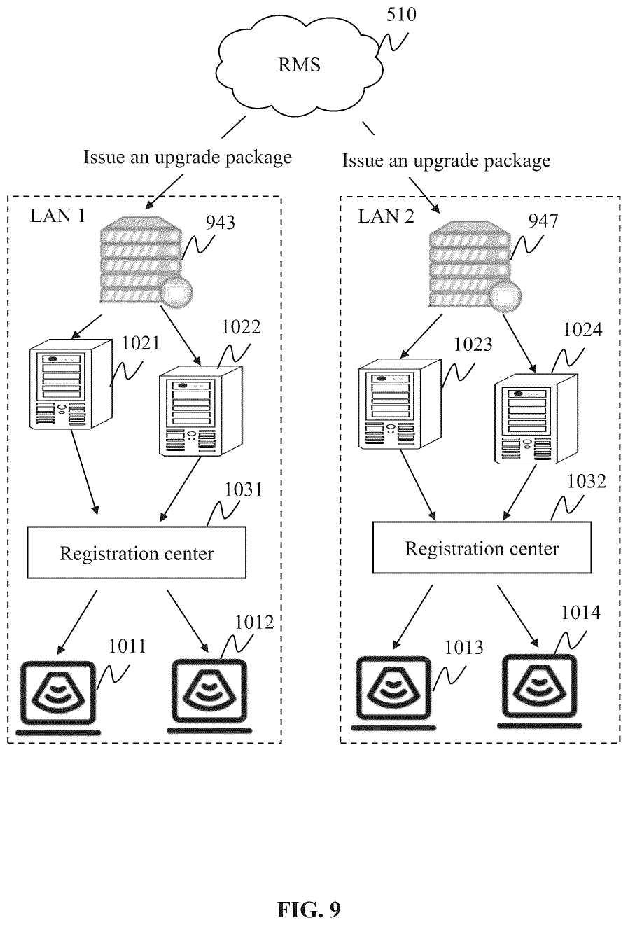

In some embodiments, each registration center 103 is connected to the medical device 101 and the at least one upgrade service module deployed on the upgrade server 102. In some embodiments, each registration center and an upgrade service module and a medical device 101 to which the registration center is connected may be deployed within the same Local Area Network (LAN). For example, as shown in FIG. 9, a registration center 1031, an upgrade service module 1021, an upgrade service module 1022, a medical device 1011, and a medical device 1012 are all deployed within LAN 1, and a registration center 1032, an upgrade service module 1023, an upgrade service module 1024, a medical device 1013, and a medical device 1014 are deployed within LAN 2.

In some embodiments, as shown in FIG. 1, the registration center 103 and the upgrade server 102 may be deployed on different servers. For example, the registration center 103 may be deployed on a registration server, and the registration server is different from the upgrade server 102. In some embodiments, as shown in FIG. 2, the registration center 103 and the upgrade server 102 may be deployed on a single server 104.

A server in the present disclosure may be a single server or a group of servers. The group of servers may be centralized or distributed. In some embodiments, the server may be local or remote. In some embodiments, the server may be implemented on a cloud platform. For example, the cloud platform may include a private cloud, a public cloud, a hybrid cloud, a community cloud, a distributed cloud, an inter-cloud cloud, a multi-cloud, or the like or any combination thereof.

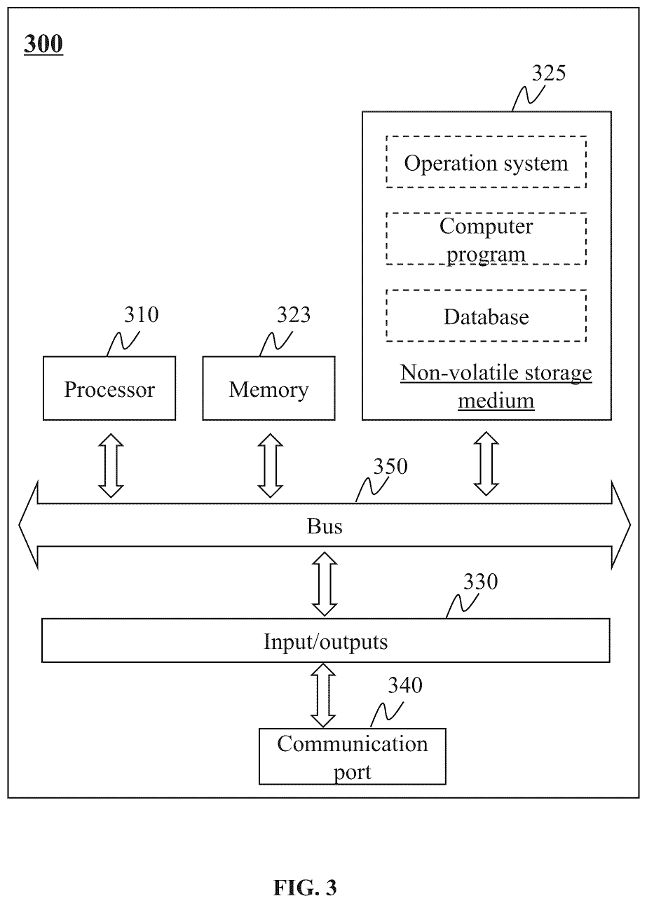

FIG. 3 is a schematic diagram illustrating exemplary software/hardware of a computing device according to some embodiments of the present disclosure. In some embodiments, a server (e.g., an upgrade server, a registration server) may be implemented by a computing device 300.

As shown in FIG. 3, the computing device 300 may include a processor 310, a storage device, inputs/outputs 330, and a communication port 340. The storage device may include a non-volatile storage medium 325 and a memory 323. The processor 310, storage device (e.g., the memory 323, the non-volatile storage medium 325), and the inputs/outputs 330 are connected via a system bus 350, and the communication port 340 is connected to the system bus 350 via the inputs/outputs 330.

The processor 310 may execute computer instructions (e.g., program code) and may perform functions of a server (e.g., a registration center, an upgrade server) in accordance with the techniques described in the present disclosure. In some embodiments, the processor 310 may include one or more hardware processors, such as a microcontroller, microprocessor, etc. For illustrative purposes only, only one processor is described in computing device 300. However, it is noted that the computing device 300 may also include a plurality of processors. Operations and/or methods described in the present disclosure that are performed by a single processor may also be performed by a plurality of processors together or separately. For example, if a processor of the computing device 300 described in the present disclosure performs an operation A and an operation B, it should be appreciated that the operation A and the operation B may also be performed by two or more different processors of the computing device 300 jointly or separately (e.g., a first processor executing the operation A and a second processor executing the operation B, or the first processor and the second processor jointly executing the operations A and B).

The storage device may store data/information obtained from the medical device 101, the upgrade server 102, the registration center 103, and/or any other component of the device upgrading system 100. For example, the non-volatile storage medium 325 may store an operating system, a computer program, and a database. The memory 323 may provide an environment for an operation of the operating system and computer programs in the non-volatile storage medium 325. The database is used to store upgrade data (e.g., an upgrade package) for the medical device 101. The processor 310 may execute the computer programs to implement a method for upgrading medical devices described herein.

The inputs/outputs 330 may be used to exchange information between the processor 310 and an external device, such as the medical device 101. In some embodiments, the input/outputs 330 may include an input device and an output device. The input device may include a keyboard, a mouse, a touch screen, a microphone, etc., or any combination thereof. The output device may include a display device, a speaker, a printer, a projector, etc. or any combination thereof.

The communication port 340 may be used to communicate with an external terminal (e.g., the medical device 101) via a network connection. The connection may be a wired connection, a wireless connection, any connection that enables data transmission and/or reception, etc., or any combination thereof.

It should be understood that the descriptions of FIGS. 1 to 3 are only provided for the purpose of illustration and do not constitute a limitation to the present disclosure. For those skilled in the art, various changes and modifications can be made under the guidance of the present disclosure. Features, structures, manners and other characteristics of embodiments of the present disclosure can be combined in various ways to obtain other and/or alternative embodiments. However, such changes and modifications do not exceed the scope of the present disclosure.

For example, the device upgrading system 100 may further include a terminal device and a network. The terminal device may interact with other components of the device upgrading system 100. In some embodiments, the terminal device may include a remote management device that is connected to the registration center 103 and/or the upgrade server(s) 102 remotely.

The remote management device may be configured to manage and/or control the registration center 103 and/or the upgrade server(s) 102. Merely by way of example, when there is a new upgrade package, a user (e.g., maintenance personnel) may upload the new upgrade package to the remote management device, and the remote management device may issue the new upgrade package to the upgrade server(s) 102 such that the upgrade server(s) 102 can upgrade multiple medical devices connected thereto. As another example, a user may set a preset wake-up time for each medical device via the remote management device. In some embodiments, the terminal device may include a user terminal connected to or integrated into a medical device 101, a user (e.g., a doctor, an operator of the medical device) may send or set information relating to the upgrade of the medical device via the user terminal. Merely by way of example, the user may set a preset wake-up time or an upgrade time for the medical device via the user terminal. The terminal device may include a mobile device, a tablet a laptop, etc., or any combination thereof. The network is used to realize an information interaction between different components. The network includes wired network and/or wireless network.

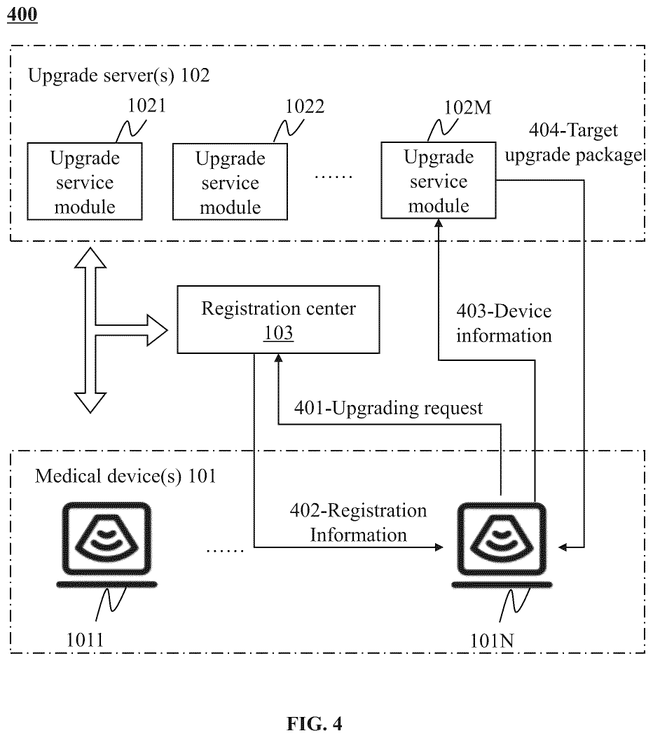

FIG. 4 is a schematic diagram illustrating an exemplary device upgrading system 400 according to some embodiments of the present disclosure. As illustrated in FIG. 4, the registration center 103 may be connected to the upgrade server(s) 102 and N medical devices 1011-101N. In some embodiments, M upgrade service modules 1021-102M may be deployed on the upgrade server(s) 102 based on a microservices framework.

The device upgrading system 400 may upgrade at least one medical device 101 by performing operations shown in FIG. 4 using the registration center 103 and the M upgrade service modules 1021-102M deployed on the upgrade server(s) 102.

Step 401, a target medical device sends an upgrade request to the registration center 103.

The upgrade request refers to a request sent by a medical device for requesting the registration center to allocate an upgrade service module for device upgrading. The target medical device is a medical device that needs to be upgraded. For example, as shown in FIG. 4, the medical device 101N is the target medical device.

In some embodiments, the target medical device may send the upgrade request to the registration center 103 at a preset time. For example, the preset time may be an automatic upgrade time of the target medical device that is preset by a user.

In some embodiments, one of the plurality of upgrade service modules may send a wake-up message to the target medical device based on a preset wake-up time. For illustration purposes, the upgrade service module that sends the wake-up message is referred to as a second target upgrade service module hereinafter. The second target upgrade service module can be any one of the upgrade service modules. For example, the second target upgrade service module may send the wake-up message to the target medical device when the preset wake-up time is reached. In response to the wake-up message, the target medical device may send the upgrade request to the registration center 103.

In some embodiments, the preset wake-up time is a time for waking up the target medical device for device upgrading. In some embodiments, the wake-up time may be preset by a user. In some embodiments, the preset wake-up time may be during non-working hours of the target medical device (e.g., nighttime, etc.).

In some embodiments, the preset wake-up time of the target medical device may be stored in the second target upgrade service module or the upgrade server(s) 102. In some embodiments, the preset wake-up time of the target medical device may be determined based on device information reported by the target medical device. For example, the user may preset the wake-up time of the target medical device based on the working hours of the target medical device, and the target medical device may report the preset wake-up time to the second target upgrade service module or the upgrade server(s) 102 as part of the device information. More information about the device information can be found in step 403.

In some embodiments, the second target upgrade service module may obtain the preset wake-up time from a terminal device. For example, the user may set the preset wake-up time for one or more medical devices via the terminal device, and the terminal device may send the preset wake-up time and a corresponding device identifier to the upgrade service modules or the upgrade server(s) 102.

In some embodiments, the second target upgrade service module may determine the preset wake-up time based on a patch package issued by a remote management device. For example, based on the patch package, the second target upgrade service module may determine a list of medical devices and their respective identifiers and preset wake-up times.

In some embodiments, the second target upgrade service module may use a WOL (Wake On LAN) technique to wake up the target medical device. The WOL technology uses a LAN device to send a command to a network card of a device that is in a standby state (e.g., hibernation) or a shutdown state, so that the device can be woken up from the standby or shutdown state to a power-on state. In some embodiments, the WOL function may be realized through a hardware module. For example, for an ultrasound device, the user may press a shortcut key DELETE when the device is powered on to enter a BIOS setup program, find the WOL setting item in the setup program, set a status of WOL to “Enable” to enable the WOL function.

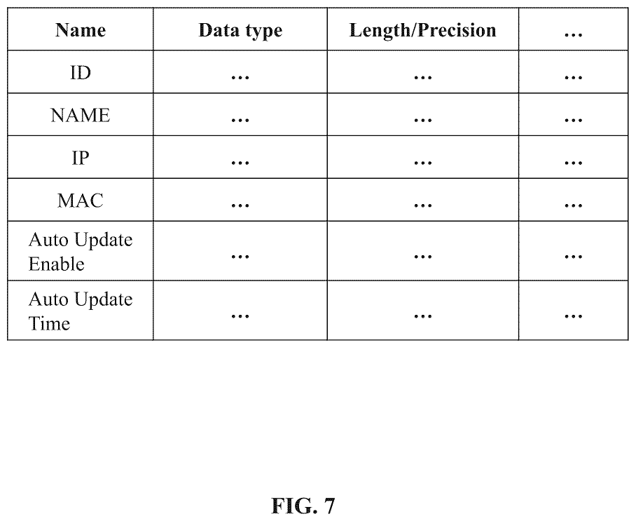

For example, the wake-up process of the target medical device may comprise the following steps: step1, the user shuts down the target medical device after using the target medical device, and the target medical device enters into a shutdown state and is in a non-powered-off state; step2, maintenance personnel remotely sends an upgrade package to an upgrade server; step3, an upgrade service module (such as the second target upgrade service module) sends a wake-up magic package, i.e., a UDP (User Datagram Protocol) package, to a local area network (LAN) at a preset wake-up time corresponding to the target medical device; the wake-up magic package is expressed in hexadecimal as 6 pairs of “FF” prefix+16 times repeated MAC address; step4, after a network card of the target medical device receives the wake-up magic package, the target medical device may be automatically powered on first, then start BIOS (Basic Input Output System), and then start an operating system. More descriptions about the wake-up of the target medical device can be found in FIG. 6.

In some embodiments, the second target upgrade service module may be any one of a plurality of upgrade service modules. The second target upgrade service module may be the same as or different from the target upgrade service module. In some embodiment, the second upgrade service module may be determined based on load information of each upgrade service module before the wake-up message is sent. For example, the second target upgrade service module may be an upgrade service module with the smallest load before the wake-up message is sent. More information about the load information of an upgrade service module can be found in operation 402.

It should be noted that the foregoing description is for illustrative purposes only and is not intended to limit the scope of the present disclosure. For a person of ordinary skill in the art, various variations and modifications may be made in accordance with the present disclosure. However, these variations and modifications do not depart from the scope of the present disclosure. For example, step 2 in the wake-up process described above may be performed before step 1.

In some embodiments, when the target medical device is in a shutdown state or a standby state at the time of receiving the wake-up message, the target medical device may power up and send the upgrade request to the registration center. For example, if the target medical device is in the shutdown state at the time of receiving the wake-up message, the target medical device may perform an automatic power-on, and after the target medical device is powered on, the target medical device may send the upgrade request to the registration center to request the registration center to assign a target upgrade service module.

In some embodiments, when the target medical device is in a working state at the time of receiving the wake-up message, the target medical device may send the upgrade request to the registration center after finishing a current workflow. For example, when the target medical device is in a process of performing a medical scan at the time of receiving the wake-up message, the target medical device may send the upgrade request to the registration center after finishing the current scan.

In some embodiments, if the target medical device is in the working state at the time of receiving the wake-up message, the target medical device may issue an upgrade time setting instruction to a terminal device. For example, the target medical device may issue the upgrade time setting instruction to the terminal device by sending an information prompt (such as a pop-up window, a short message, etc.), an audible prompt (such as a voice prompt, a ringing prompt, etc.), or the like. In some embodiments, the target medical device may receive an upgrade time from the terminal device, and send the upgrade request to the registration center 103 at the upgrade time. For example, a user (e.g., an operator of the medical device or maintenance personnel) may enter the upgrade time for the target medical device to perform an automatic upgrade via a terminal device. The target medical device may receive the upgrade time and send the upgrade request to the registration center 103 connected to the target medical device (e.g., the registration center 103 that is within the same local area network as the target medical device) when reaching the upgrade time.

In some embodiments, an upgrade service module sends the wake-up message to the target medical device according to the preset wake-up time, and the target medical device delays upgrading when it is in the working state at the time of receiving the wake-up message, and sends the upgrade request to the registration center 103 when it is in a non-working state. In this way, upgrade and maintenance of the target medical device can be carried out during non-working hours of the target upgrade service module, therefore avoiding affecting the normal operation of the target medical device.

Step 402, in response to the upgrade request sent by the target medical device, the registration center 103 may determine the target upgrade service module from a plurality of upgrade service modules (e.g., an upgrade service module 1021, an upgrade service module 1022, . . . , an upgrade service module 102M), and send registration information of the target upgrade service module to the target medical device.

Registration information of an upgrade service module may include address information of the upgrade service module, for example, an IP address and a port number. In some embodiments, before receiving the upgrade request sent by the target medical device, the registration center 103 may receive registration information sent by the plurality of upgrade service modules and store the registration information. Specifically, each upgrade service module connected to the registration center 103, after being powered on, may send its registration information to the registration center 103 for registration. The registration center 103 may store registration information sent by each upgrade service module. For example, in conjunction with FIG. 4, the upgrade service module 1021, the upgrade service module 1022, . . . , the upgrade service module 102M, after being powered on, may send their IP addresses and port numbers to the registration center 103 for registration, and the registration center 103 may store the IP addresses and the port numbers of M upgrade service modules (e.g., store the IP addresses and port numbers in a storage media of the registration center 103). Based on the registration information of each upgrade service module, the registration center 103 may effectively manage each upgrade service module based on the microservices framework.

In some embodiments, for each upgrade service module, the registration center 103 may send a heartbeat signal to the upgrade service module, and determine a connection state between the upgrade service module and the registration center 103 based on a response of the upgrade service module to the heartbeat signal. Further, in response to determining that the connection state is a disconnected state, the registration center 103 may delete the registration information of the upgrade service module. The heartbeat signal (or a heartbeat package) is a periodic signal generated by the registration center 103 to detect whether a connection to an upgrade service module is normal. In some embodiments, the heartbeat signal may be a package sent regularly. The registration center 103 sends the heartbeat signal to the upgrade service module regularly, the upgrade service module needs to respond to the heartbeat signal after receiving the heartbeat signal. If the heartbeat signal fails to be sent to the upgrade service module or no response is received from the upgrade service module for a long period, there may be a problem in the connection between the registration center 103 and the upgrade service module. For example, in conjunction with FIG. 4, the registration center 103 may send a heartbeat signal in real time or at a specific time interval to the upgrade service module 1021, the upgrade service module 1022, . . . , and the upgrade service module 102M, respectively, and determine a connection state between each upgrade service module and the registration center 103 based on a response of each upgrade service module to the heartbeat signal. For example, if the registration center 103 detects that the upgrade service module 1022 is disconnected, it may delete registration information of the upgrade service module 1022 from a storage device of the registration center 103.

By setting a heartbeat detection mechanism for monitoring the connection state of each upgrade service module and deleting the registration information of an upgrade service module when the upgrade service module is disconnected from the registration center 103, the registration center 103 may only store registration information of all available upgrade service modules.

The target upgrade service module refers to an upgrade service module that provides upgrade service for the target medical device selected from a plurality of upgrade service modules.

In some embodiments, the registration center 103 may determine the target upgrade service module from the plurality of upgrade service modules based on first load information of each upgrade service module. The first load information of an upgrade service module may reflect a count of medical devices served by the upgrade service module currently. The load of an upgrade service module may be greater if it is serving a greater count of medical devices.

In some embodiments, the registration center 103 may determine the target upgrade service module from the plurality of upgrade service modules based on a preset policy and the first load information of each upgrade service module. The preset policy may be, but is not limited to, a load balancing policy or a polling policy.

For example, if an upgrade service module A and an upgrade service module B are deployed on the upgrade server 102, and the upgrade service module A is currently providing an upgrade service for two medical devices, and the upgrade service module B is currently providing an upgrade service for four medical devices, then the registration center 103 may record a load of the upgrade service module A is 2 and a load of the upgrade service module B iis 4. According to the load balancing policy, due to a smaller load of the upgrade service module A, when there is a new medical device (e.g., a target medical device) that needs to be assigned with an upgrade service module, the registration center 103 may determine the upgrade service module A as a target upgrade service module and assign the upgrade service module A to the new medical device.

As another example, when both the upgrade service module A and upgrade service module B are currently providing the upgrade service for 2 medical devices, the registration center 103 may record that the load of both upgrade service modules A and B is 2, i.e., they are equally loaded. In such cases, the registration center 103 may assign the upgrade service module A and the upgrade service module B to a new medical device in turn based on the polling policy. For example, the registration center 103 may assign the upgrade service module A to a target medical device and assign the upgrade service module B to a next target medical device.

In this embodiment, the registration center 103 automatically selects the target upgrade service module from the plurality of upgrade service modules based on the load of each upgrade service module (the first load information), henceforth automatically providing the upgrade service for the target medical device, achieving load balancing, and improving the efficiency of upgrading medical devices.

In some embodiments, the plurality of upgrade service modules may be deployed on a plurality of upgrade servers, each upgrade service module may be deployed on one of the upgrade servers. For each upgrade service module, the registration center 103 may obtain the first load information of the upgrade service module and second load information of an upgrade server on which the upgrade service module is deployed. Further, the registration center 103 may determine, based on the first load information and the second load information corresponding to each upgrade service module, the target upgrade service module from the plurality of upgrade service modules. The second load information corresponding to an upgrade service module may reflect a current data processing amount of services currently provided by the upgrade server on which the upgrade service module is deployed. The services provided by the upgrade server may include an upgrade service, a business service (e.g., a service such as fault detection, device maintenance, etc.), etc. The more services the upgrading server provides, the greater the data processing amount and the greater the load.

In some embodiments, the registration center 103 may determine a target upgrade server from a plurality of upgrade servers based on the load balancing policy and the second load information of the plurality of upgrade servers. For example, the target upgrade server may be a server with the smallest load among the plurality of upgrade servers. Further, the target upgrade service module may be determined from a plurality of upgrade service modules deployed on the target upgrade server based on the first load information of each upgrade service module. For example, the target upgrade service module may be an upgrade service module with the smallest load deployed on the target upgrade server.

In some embodiments, when there is a plurality of upgrade servers, the registration center 103 may determine a server list based on a weight value of each upgrade server, and determine the target upgrade server from the server list based on a numerical index. The weight value of an upgrade server may reflect the ability of the upgrade server to provide the upgrade service. The weight value of the upgrade server may be a preset value. The weight value may be related to information such as processor performance, memory size, etc., of the upgrade server. Optionally, the preset value may be adjusted based on the second load information of the upgrade server and the adjusted preset value may be designated as the weight value of the upgrade server.

The server list includes a plurality of identifiers of the upgrade servers. Exemplarily, a count of occurrences of the identifier of each upgrade server in the server list is related to the weight value of the upgrade server. The higher the weight value, the higher the count of occurrences. It is assumed that there are m upgrade servers denoted as S={S0, S1, S2, . . . , Sm}, and the preset weights of the m upgrade servers are W={W0, W1, W2, . . . , Wm}, and a sum of the preset weights of the m upgrade servers is denoted as weight-Sum. The registration center 103 may add N0 identifiers of the upgrade server S0 (i.e., the count of occurrences of the upgrade server S0 is N0), N1 identifiers of the upgrade server S1, N2 identifiers of the upgrade server S2, . . . , Nm identifiers of the upgrade server Sm to the list to form the server list. Ni is positively correlated to Wi. The identifiers of the upgrade servers in the server list may be ranked randomly or in a specific order.

In some embodiments, the registration center 103 may generate the numerical index based on a random function. For example, the registration center may generate any integer between 0 and the sum of N0 to Nm by the random function, use the integer as the numerical index to obtain a corresponding upgrade server from the server list and determine the obtained upgrade server as the target upgrade server. Merely by way of example, if the sum of N0 to Nm is 100, a random value in the range of 1-100 may be determined using the random function, and the upgrade server whose serial number is the random value may be determined from the server list as the target upgrade server.

In some embodiments, the registration center 103 may determine the target upgrade service module from the plurality of upgrade service modules deployed on the target upgrade server based on the first load information of each upgrade service module. For example, the target upgrade service module may be determined from the plurality of upgrade service modules deployed on the target upgrade server based on the load balancing policy or the polling policy.

In some embodiments, when the registration center 103 receives an upgrade request from a plurality of target medical devices at the same time, it may determine a corresponding target upgrade service module for each target medical device based on a priority ranking of the target medical devices. Specifically, the registration center 103 may determine a priority ranking of the target medical devices based on device information of the target medical devices; based on the priority ranking of the target medical devices, the registration center 103 determines a corresponding target upgrade service module for each of the target medical devices in turn.

In some embodiments, the registration center 103 may determine the priority ranking of the target medical devices based on version information of the target medical devices. In some embodiments, the registration center 103 may determine the priority ranking of the target medical devices based on business information of the target medical devices. In some embodiments, the registration center 103 may determine the priority ranking of the target medical devices based on both the version information and business information of the target medical devices.

The version information may reflect a current version number of software in a target medical device. For example, the current version number of the software may include V1.0.1, V1.0.2, V1.0.3, or V1.0.4, etc. The higher the current version of the target medical device, the closer it is to the target version (e.g., the latest version of the software), the less time it takes to upgrade, and the higher its priority ranking may be. For example, a target medical device with a current version of V1.0.3 may be ranked before a target medical device with a current version of V1.0.1, and the registration center 103 may assign a target upgrade service module to the target medical device with a current version of V1.0.3 first. Or, considering that a target medical device of a lower version may have a more urgent need to be upgraded, the target medical device having a lower version may have a higher priority ranking.

The business information may reflect a workload of a target medical device during a certain period (e.g., the last week, the last month, the last 15 days, etc.). For example, the workload of the target medical device during the most recent week may include 525 hours, 340 hours, 788 hours, etc. The lower the workload of the target medical device, the higher the priority ranking of the target medical device.

In some embodiments, the registration center 103 may obtain the version information and/or business information sent in real-time by a target medical device.

In some embodiments, the registration center 103 may determine an initial priority ranking of the target medical devices based on the version information of the target medical devices; and adjust the initial priority ranking based on the business information of the target medical devices to determine the priority ranking of the target medical devices. Exemplarily, after filtering target medical devices with the latest version, the registration center 103 may rank remaining target medical devices in a high-to-low order or a low-to-high order based on the current versions of the remaining target medical devices to determine the initial priority ranking. When there exists a plurality of target medical devices with the same current version, the registration center 103 may further rank the target medical devices with the same current version according to an amount of business (i.e., the workload) of the target medical devices.

For example, if it is assumed that five ultrasound devices {US1, US2, US3, US4, US5} simultaneously send an upgrade request to the registration center 103, the current versions of the five ultrasound devices are {v1.0.0, v4.0.0, v2.0.0, v3.0.0, v1.0.0}, and the business volumes of the five ultrasound devices in the last week are {525, 340, 788, 214, 462}, and an upgrade package for the latest version V5.0.0 exists on the upgrade server. The registration center 103 may sort the 5 ultrasound devices in a descending order based on version information of the 5 ultrasound devices, and an initial priority ranking may be {US2, US4, US3, US1 or US5}. Because US1 and US5 have a same version number, the registration center 103 may further rank US1 and US5 based on the business volumes of the US1 and US5, and the priority ranking may be {US2, US4, US3, US5, US1}. The registration center 103 may determine the target upgrade service module for each of the ultrasound devices in turn based on the priority ranking list {US2, US4, US3, US5, US1}.

By prioritizing the target medical devices, the overall efficiency of the upgrade service can be improved. For example, target medical devices with more urgent upgrade needs can be prioritized to receive upgrade services based on the version information. As another example, idle target medical devices can be prioritized to receive upgrade services based on the business information.

Step 403, the target medical device establishes a communication connection with the target upgrade service module based on the registration information of the target upgrade service module and sends device information to the target upgrade service module through the communication connection.

The device information of a medical device may include any information related to the medical device. In some embodiments, the device information may include a device identifier of the medical device (e.g., the target medical device), a device name, a device IP address, a device MAC (Media Access Control), the version information, the business information, and an automatic upgrade switch (i.e., whether an automatic upgrade is turned on), an automatic upgrade time, or the like, or any combination thereof, of the medical device.

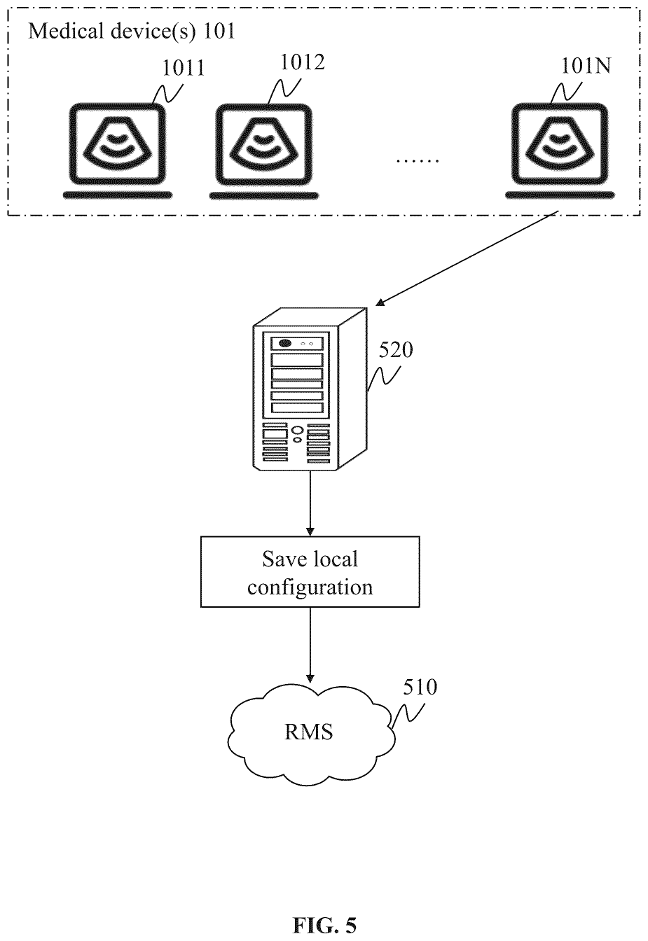

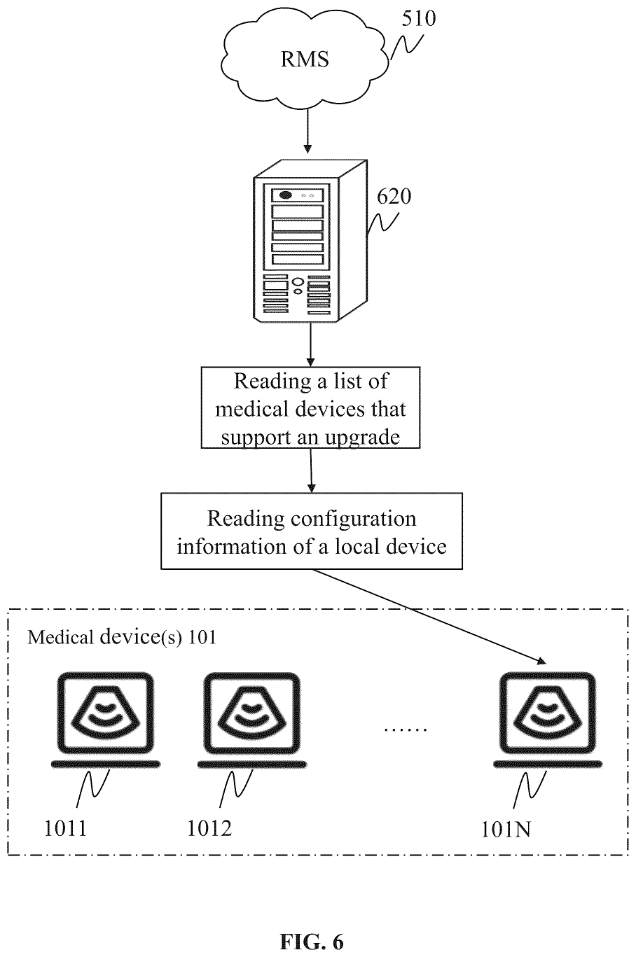

In some embodiments, the target medical device may report the device information to the target upgrade service module via an RPC (Remote Procedure Call) request. In some embodiments, an upgrade client may be installed on the target medical device or a terminal device connected to the target medical device, and the upgrade client starts up when the target medical device is powered on. After the upgrade client starts up, the upgrade request may be automatically sent to the registration center 103 connected to the target medical device to obtain the registration information of the target upgrade service module. After the upgrade client establishes a communication connection with the target upgrade service module based on the registration information, the upgrade client may report the device information to the target upgrade service module through the communication connection. More descriptions about the report of the device information can be found in FIG. 5.

In some embodiments, the target medical device may send the device information to the upgrade server 102. For example, after the target medical device is activated, the device information may be reported to the upgrade server 102 that establishes a communication connection with the target medical device.

By reporting the device information to the target upgrade service module or the upgrade server 102, it can ensure that the latest device information of the target medical device is always available to the target upgrade service module.

Step 404, the target upgrade service module determines a target upgrade package matching the target medical device based on the device information and sends the target upgrade package to the target medical device.

The target upgrade package may include packaged upgrade data used to upgrade the target medical device. The target upgrade package may be sent to the target medical device through the communication connection between the target upgrade service module and the target medical device.

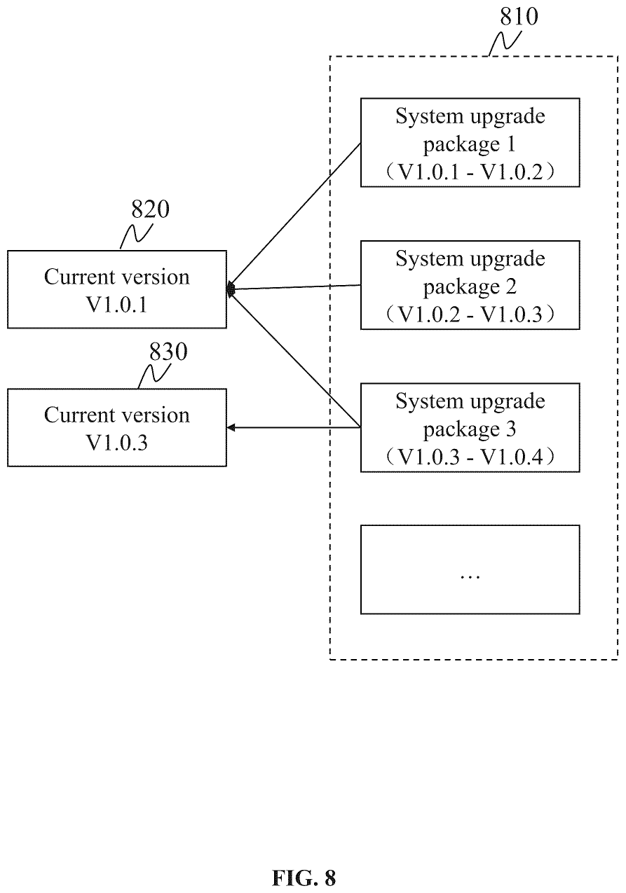

In some embodiments, an upgrade service module (e.g., the target upgrade service module) or the upgrade server (e.g., the upgrade server on which the target upgrade service module is deployed) may store the device information. More descriptions about the storage of the device information can be found in FIG. 7.

In some embodiments, the target upgrade service module may determine a current version of the target medical device based on version information in the device information, and compare the current version with a target version corresponding to the target upgrade service module to determine the target upgrade package. Specifically, in response to determining that the current version of the target medical device is lower than the target version, the target upgrade service module may determine an upgrade link from the current version to the target version. Further, based on the upgrade link, a target upgrade package matching the target medical device is determined from at least one upgrade package of the target upgrade service module. If the current version of the target medical device is not lower than the target version, no upgrade is required for the target medical device.

The current version is represented by a current version number of a software in the target medical device. The target version is represented by a latest version number of a software in the target upgrade service module. The upgrade link refers to an upgrade path that goes through from an initial version (e.g., the current version) to an ending version (e.g., the target version). For example, V1.0.1 is the initial version and V1.0.4 is the ending version, then an upgrade link from the initial version to the ending version may be V1.0.1-V1.0.2-V1.0.3-V1.0.4. In some embodiments, the upgrade link may be determined based on a version upgrading record. For example, if V1.0.0 is the initial version and V1.0.4 is the ending version, the initial version is first upgraded into version V1.0.2, and then upgraded into version V1.0.4, the upgrade link may be V1.0.0-V1.0.2-V1.0.4.

In some embodiments, the target upgrade package may include description information of the target upgrade package, the description information includes an initial version (From Version) and an ending version (To Version or the target version) corresponding to the target upgrade package, indicating that the target upgrade package may be used to upgrade software from the initial version to the ending version. In some embodiments, the target upgrade package may include one or more upgrade packages. More information about the target upgrade package can be found in FIG. 8.

In this embodiment, when the current version of the target medical device is lower than the target version corresponding to the target upgrade service module, the upgrade link and the target upgrade package matching the medical device can be determined automatically, henceforth realizing an automatic upgrade of the target medical device and improving the upgrade efficiency of the medical device.

In some embodiments, the at least one upgrade package in the target upgrade service module is from a remote management device. The remote management device may be an RMS (Remote Management Service) device. For example, prior to receiving an upgrade request from the target medical device, each upgrade service module may receive at least one upgrade package sent by the RMS device through a gateway.

The gateway, the registration center, and the upgrade service modules are all components of the microservices framework. Exemplarily, as shown in FIG. 9, a gateway 947 may also be deployed within a local area network 2 (LAN2) where upgrade service modules 1023 and 1024, a registration center 1032, and medical devices 1013, 1014 are located, and the upgrade service modules 1023 and 1024 are connected to the registration center 1032 and the gateway 947, respectively. The RMS device may send a plurality of upgrade packages to the gateway, then the gateway distributes the plurality of upgrade packages to each upgrade service module within the same LAN as the gateway is located. In some embodiments, the gateway may send the upgrade package received from the remote management device to an upgrade server. Each upgrade service module may read the upgrade package from the upgrade server on which the upgrade service module is deployed.

In some embodiments, the gateway may be software deployed on the same server as the upgrade service modules and/or the registration center. In some embodiments, the gateway and the registration center may be deployed on one server (e.g., the registration server 103), and the upgrade service modules deployed on one or more additional servers (e.g., the upgrade server 102). In some embodiments, the gateway, the registration center, and the upgrade service modules may be deployed on the same server (e.g., the server 104). In some embodiments, two or more upgrade service modules may access a public network through the same gateway. The gateway may uniformly receive upgrade packages sent by the RMS device and forward the upgrade packages to each upgrade service module to which it is connected after identity authentication and protocol conversion.

In some embodiments, upgrade data (such as the upgrade package) is sent to the upgrade service module in a batch via the remote management device, so that the upgrade service module can upgrade a plurality of medical devices in a batch based on the upgrade data, which improves the efficiency of upgrading medical devices.

In some embodiments, the target upgrade service module may obtain an upgrade result of the target medical device and send the upgrade result to the remote management device via the gateway.

After receiving the target upgrade package sent by the target upgrade service module, the target medical device may upgrade based on the target upgrade package. The upgrade result may reflect a normal upgrade or an abnormal upgrade of the target medical device. Exemplarily, if the target medical device is upgraded normally, a normal upgrade result is generated and fed back to the target upgrade service module. The target upgrade service module may report the normal upgrade result to the RMS device through the gateway. If the target medical device is upgraded abnormally, an abnormal upgrade result is generated and fed back to the target upgrade service module, then the target upgrade service module reports the abnormal upgrade result to the RMS device through the gateway.

By enabling the target upgrade service module to receive the upgrade result of the target medical device and send the upgrade result to the remote management device through the gateway, a user can timely know whether the upgrade of the target medical device is normal or not, which ensures that upgrade abnormality can be handled timely.

For illustration purposes, a complete upgrade process of an ultrasound device including the following steps is described below. An upgrade client installed on the ultrasound device may be a Daemon service of a Linux system.

Step 1, when the Linux system is powered on, the Daemon service runs automatically after the startup of the ultrasound device. Thus, when the ultrasound device boots into the Linux system, the upgrade client program also starts.

Step 2, after the upgrade client starts, the upgrade client sends an upgrade request to a connected registration center to obtain an address of an upgrade service module. The registration center may determine a target upgrade service module based on a load balancing policy or a polling policy and send an IP address of the target upgrade service module to the upgrade client. The upgrade client proactively establishes a connection with the target upgrade service module based on the IP address and reports its device information to the target upgrade service module.

Step 3, the target upgrade service module determines whether the ultrasound device needs to be upgraded. If the ultrasound device needs to be upgraded, the target upgrade service module determines a target upgrade package and issues the target upgrade package to the ultrasound device. The upgrade client is notified to perform an upgrade.

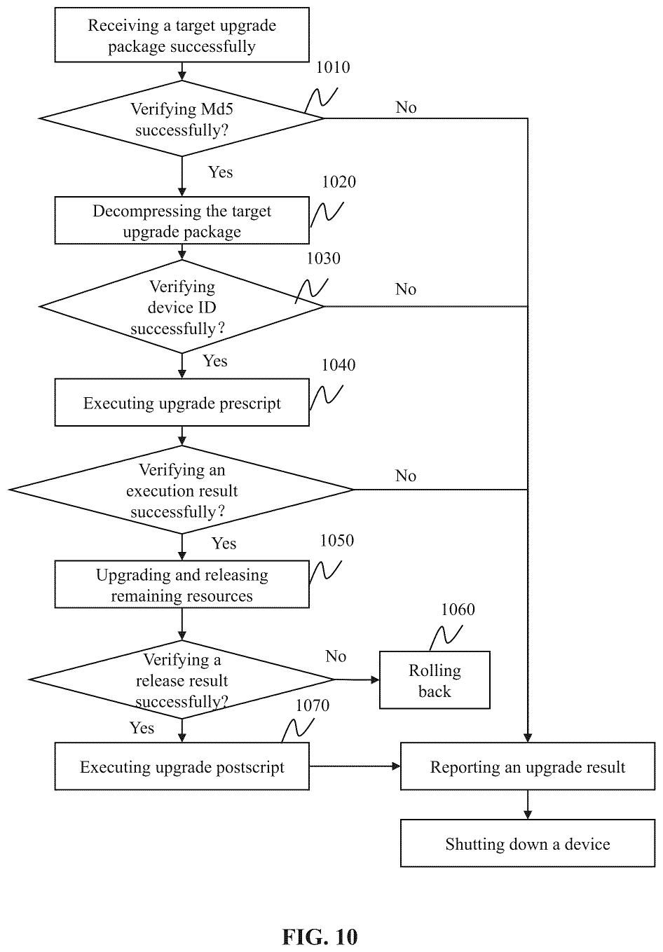

Step 4, upon receiving an upgrade notification and the target upgrade package, the upgrade client performs an automatic upgrade in accordance with an upgrade process. As shown in FIG. 10, the upgrade process may include unpacking the target upgrade package, executing an upgrade prescript, releasing remaining resources, and executing an upgrade postscript. More descriptions about the upgrade process can be found in FIG. 10.

Step 5, the upgrade client completes the upgrade, actively reports an upgrade result to the target upgrade service module, and automatically shuts down after the report is completed.

The above upgrade process of the upgrade client of the ultrasound device may be performed based on a microservices framework, wherein a plurality of upgrade service modules is deployed in a local area network (the plurality of upgrade service modules is deployed on one or more upgrade servers), and each upgrade service module may provide an upgrade service to a plurality of medical devices within the same LAN. As a result, whenever the upgrade service modules detect that there is an upgrade task, they can perform upgrade services on the plurality of medical devices within the current LAN; at the same time, the remote terminal platform can also send the upgrade package to a plurality of hospitals and/or a plurality of LANs, thus achieving upgrading multiple medical devices simultaneously.

FIG. 5 is a schematic diagram illustrating an exemplary process for reporting device information of a target medical device according to some embodiments of the present disclosure.

As shown in FIG. 5, a medical device 101N is the target medical device. After an upgrade client installed on the target medical device establishes a communication connection with a corresponding target upgrade service module 520 based on registration information (e.g., an IP address, a port number) of the target upgrade service module 520, the upgrade client may actively report device information to the target upgrade service module 520 through the communication connection. The device information may include a device identifier, a device name, a device IP address, a device MAC, version information, business information, an automatic upgrade switch, and an automatic upgrade time, etc. The target upgrade service module 520 may save a local configuration based on the device information and report the device information to an RMS device 510 through the Hypertext Transfer Protocol Secure (HTTPS) Protocol. The RMS device may be a terminal or a server on the public network.

FIG. 6 is a schematic diagram illustrating an exemplary wake-up process of a medical device according to some embodiments of the present disclosure.

As shown in FIG. 6, in some embodiments, a second target upgrade service module 620 (or an upgrade server where the second target upgrade service module 620 is located) may receive a patch package (e.g., an upgrade package) remotely issued by a user (e.g., operation and maintenance personnel) via the RMS device 510. The patch package may include a resource file for upgrading, a list of devices that need to be upgraded (e.g., an identifier of a medical device that needs to be upgraded), encrypted data (e.g., a patch package md5 value), and other information. Further, the second target upgrade service module 620 may read the list of devices that need to be upgraded from the patch package and read configuration information of the medical devices. The configuration information of a medical device may include but is not limited to, an identifier and a preset wake-up time of the medical device. The second target upgrade service module 620 may perform a timed wake-up task based on configuration information of medical devices in the list of medical devices. For example, if an identifier of the target medical device 101N is in the list of medical devices, when a preset wake-up time for the target medical device 101N reaches, the second target upgrade service module 620 may generate a wake-up magic package and send the wake-up magic package to the target medical device 101N.

FIG. 7 is a schematic diagram illustrating exemplary device information stored in an upgrade service module according to some embodiments of the present disclosure. As shown in FIG. 7, a device information table is stored in a database of an upgrade service module or an upgrade server, the device information table includes a device identifier, a device name, a device IP address, a device MAC, an automatic upgrade switch, and an automatic upgrade time of each medical device. In some embodiments, the device information table may include information like Data type, Length/Precision, etc. of each piece of information.

FIG. 8 is a schematic diagram illustrating an exemplary process for determining an upgrade link according to some embodiments of the present disclosure. As shown in FIG. 8, an upgrade service module 810 (e.g., a target upgrade service module) may store historical upgrade package data. The upgrade service module 810 may automatically match a target upgrade package matching a target medical device based on a current version of the target medical device (a target medical device 820, a target medical device 830) and a latest version (i.e., a target version) in the upgrade service module 810. As shown in FIG. 8, a current version of the target medical device 820 is V1.0.1, a current version of the target medical device 830 is V1.0.3, and a target version of the upgrade service module 810 is V1.0.4. For the target medical device 820, the upgrade service module 810 may take V1.0.1 as an initial version and V1.0.4 as an ending version and determine an upgrade link V1.0.1-V1.0.2-V1.0.3-V1.0.4 from the initial version V1.0.1 to the ending version V1.0.4, determine system upgrade packages corresponding to the upgrade link. The upgrade packages corresponding to the upgrade link include a system upgrade package 1 (from V1.0.1 to V1.0.2), a system upgrade package 2 (from V1.0.2 to V1.0.3), and a system upgrade package 3 (from V1.0.3 to V1.0.4). The system upgrade package 1, the system upgrade package 2, and the system upgrade package 3 may be combined as a target upgrade package for the target medical device 820. For the target medical device 830, the upgrade service module 810 may take V1.0.3 as an initial version and V1.0.4 as an ending version and determine an upgrade link V1.0.3-V1.0.4 from the initial version V1.0.3 to the ending version V1.0.4, and determine a system upgrade package 3 (from V1.0.3 to V1.0.4) as a target upgrade package for the target medical device 830, and then issue the target upgrade package to the medical device 830.

FIG. 9 is a schematic diagram illustrating an exemplary process of issuing an upgrade package according to some embodiments of the present disclosure.

As shown in FIG. 9, an RMS device 510 may be deployed in a WAN (Wide Area Network) network, a registration center 1031, an upgrade service module 1021, an upgrade service module 1022, and a gateway 943 may be deployed in LAN 1 where medical devices 1011 and 1012 are located; and a registration center 1032, an upgrade service module 1023, an upgrade service module 1024, and a gateway 947 may be deployed in LAN 2 where medical devices 1013 and 1014 are located. The registration center, the upgrade service modules, and the gateway located in the same LAN may be deployed in the same server (e.g., the server 104) or different servers. For example, the registration center 1031 and the gateway 943 are deployed on a registration server, and the upgrade service module 1021 and the upgrade service module 1023 are deployed on an upgrade server. As another example, the registration center 1031, the upgrade service module 1021, the upgrade service module 1022, and the gateway 943 are deployed on the same server (e.g., the server 104).

The user may issue one or more upgrade packages through the RMS device 510, the one or more upgrade packages are transmitted via a gateway to each upgrade service module, and each upgrade service module may use the upgrade packages to upgrade the corresponding medical device(s).

FIG. 10 is a schematic diagram illustrating an exemplary process of upgrading a target medical device according to some embodiments of the present disclosure.

As shown in FIG. 10, after receiving a target upgrade package, a target medical device executes the following steps based on the target upgrade package.

Step 1010, the target medical device performs a md5 verification on the target upgrade package. If the md5 verification fails, an abnormal upgrade result is generated; if the md5 verification passes, step 1020 is performed.

Step 1020, the target medical device decompresses the target upgrade package.

Step 1030, the target medical device verifies device ID based on the decompressed target upgrade package. If the device ID is verified unsuccessfully, an abnormal upgrade result is generated; if the device ID is verified successfully, step 1040 is performed.

Step 1040, the target medical device executes an upgrade prescript corresponding to the target upgrade package. The target medical device may then verify an execution result of the prescript. If the verification fails, an abnormal upgrade result is generated; if the verification passes, step 1050 is performed.

Step 1050, the target medical device is upgraded according to the target upgrade package, and releases the remaining resources. The remaining resources are surplus resources used in the process of upgrading. The target medical device may verify a release result, if the verification fails, an abnormal upgrade result is generated and step 1060 is performed to roll back; if the verification passes, step 1070 is performed.

Step 1070, the target medical device executes an upgrade postscript, generates a normal upgrade result after finishing the execution. Lastly, the target medical device may report the abnormal upgrade result or the normal upgrade result to a remote management device via a target upgrade service module and automatically shut down.

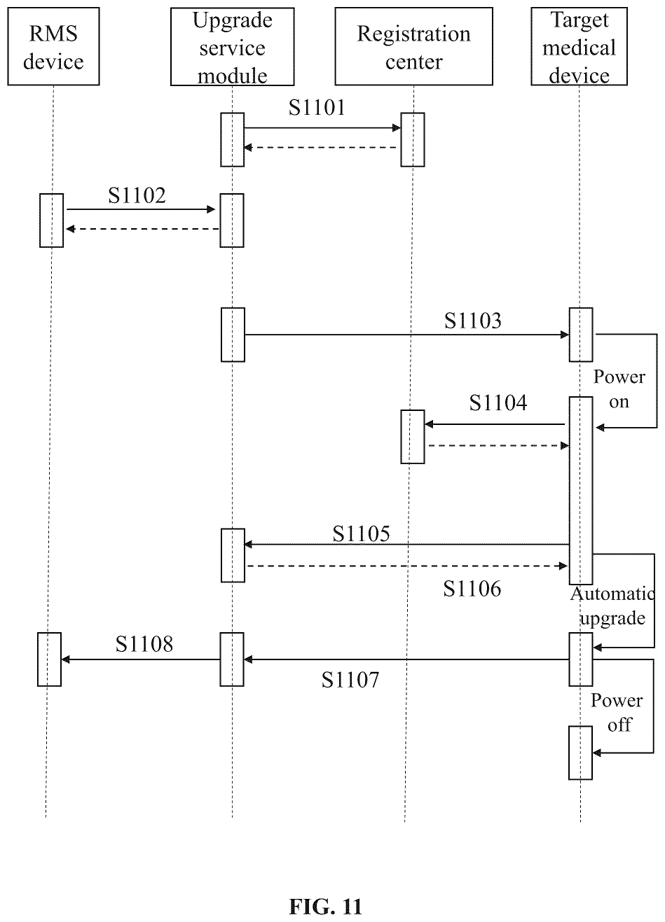

FIG. 11 is a schematic diagram illustrating an exemplary process of upgrading a target medical device according to some embodiments of the present disclosure. As shown in FIG. 11, the upgrade process based on a microservices framework may include the following steps.

S1101, after each upgrade service module is powered on, each upgrade service module sends registration information to a registration center for registration. The registration information may include an IP address and a port number of each upgrade service module. The registration center may store the registration information.

S1102, a user sends a system upgrade package to the upgrade service modules through an RMS device.

S1103, a second target upgrade service module sends a wake-up message to the target medical device according to a preset wake-up time. The second upgrade service module may store a preset wake-up time for each medical device in advance, or may receive preset wake-up time information for each medical device in advance.

S1104, when the target medical device is in a shutdown state or a standby state at a time of receiving the wake-up message, the target medical device turns on the device based on the wake-up message. An upgrade client of the target medical device is then automatically powered on. The target medical device may send an upgrade request to the registration center via the upgrade client to request the registration center to assign an upgrade service module to the target medical device. In response to the upgrade request sent by the target medical device, the registration center may determine a target upgrade service module from the plurality of upgrade service modules and send registration information of the target upgrade service module to the target medical device.

S1105, the upgrade client of the target medical device establishes a communication connection with the target upgrade service module based on the registration information and sends device information to the target upgrade service module via the communication connection.

S1106, the target upgrade service module determines a system upgrade package for upgrading the target medical device, determines a target upgrade package, notifies the upgrade client of the target medical device to perform an upgrade and issues the target upgrade package to the upgrade client. The upgrade client receives the target upgrade package and performs a device upgrade based on the target upgrade package.

S1107, after the upgrade of the target medical device is completed, an upgrade result is reported to the target upgrade service module, and the target medical device is automatically shut down.

S1108, the upgrade service module (e.g., the target upgrade service module) forwards the upgrade result of the target medical device to the RMS device.

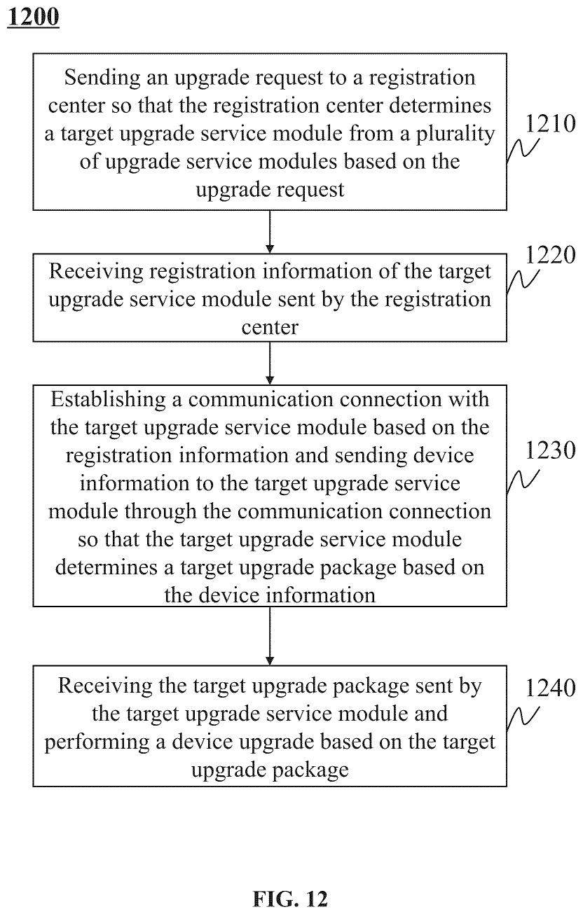

FIG. 12 is a flowchart illustrating an exemplary process for upgrading a target medical device according to some embodiments of the present disclosure. A process 1200 may be performed by a target medical device.

Step 1210, the target medical device may send an upgrade request to a registration center so that the registration center determines a target upgrade service module from a plurality of upgrade service modules based on the upgrade request.

In some embodiments, the target medical device may send the upgrade request to a registration center at a preset time. In some embodiments, the target medical device may send the upgrade request to the registration center in response to a wake-up message sent by an upgrade service module (e.g., a second target upgrade service module). In some embodiments, the registration center may determine the target upgrade service module from the plurality of upgrade service modules based on first load information of each upgrade service module. More descriptions can be found in FIG. 3.

Step 1220, the target medical device may receive registration information of the target upgrade service module sent by the registration center.

After the registration center determines the target upgrade service module, the registration center may send registration information of the target upgrade service module to the target medical device. More descriptions can be found in FIG. 3.

Step 1230, the target medical device may establish a communication connection with the target upgrade service module based on the registration information and send device information to the target upgrade service module through the communication connection so that the target upgrade service module determines a target upgrade package based on the device information.

For example, an upgrade client may be installed on the target medical device, and the upgrade client is powered on as the target medical device is powered on. In some embodiments, the upgrade client of the target medical device may establish a communication connection with the target upgrade service module based on the registration information, and send device information to the target upgrade service module through the communication connection. In some embodiments, the target upgrade service module may determine a current version of the target medical device based on version information in the device information, and compare the current version with a target version corresponding to the target upgrade service module to determine the target upgrade package. More descriptions can be found in FIG. 3.

Step 1240, the target medical device may receive the target upgrade package sent by the target upgrade service module and perform a device upgrade based on the target upgrade package.

In some embodiments, the target medical device may be automatically upgraded based on the target upgrade package if the target upgrade package is received successfully. In some embodiments, the upgrade client of the target medical device may perform an automatic upgrade according to a standard upgrade process based on the target upgrade package. In some embodiments, the target medical device may report an upgrade result (e.g., a normal upgrade result or an abnormal upgrade result) to the target upgrade service module. More descriptions can be found in FIG. 10.

In some embodiments, a number of upgrade service modules may be increased to provide upgrade services simultaneously to improve the efficiency of device upgrading. In addition, a plurality of upgrade service modules and the registration center may be deployed on the same server to avoid adding additional costs; the plurality of upgrade service modules and the registration center may also be deployed separately on multiple servers to maximize processing efficiency. That is, a method for upgrading medical devices based on a microservices framework can support flexible deployment.

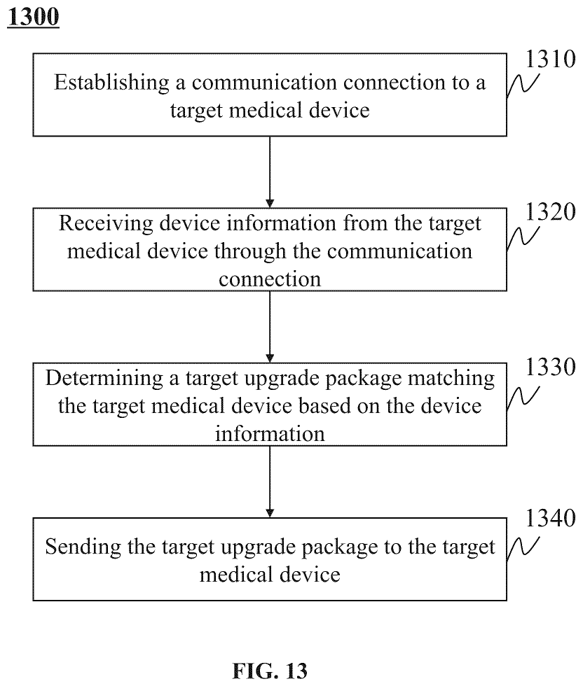

FIG. 13 is a flowchart illustrating an exemplary process for upgrading a target medical device according to some embodiments of the present disclosure. A process 1300 may be performed by a target upgrade service module. The target upgrade service module is determined from at least one upgrade service module deployed on an upgrade server by a registration center in response to an upgrade request sent by the target medical device.

Step 1310, the target upgrade service module establishes a communication connection to the target medical device.

In some embodiments, after the registration center determines the target upgrade service module, the registration center may send registration information of the target upgrade service module to the target medical device, and the target medical device establishes the communication connection to the target upgrade service module.

Step 1320, the target upgrade service module receives device information from the target medical device through the communication connection. More descriptions can be found in FIG. 4.

Step 1330, the target upgrade service module determines a target upgrade package matching the target medical device based on the device information. More descriptions can be found in FIG. 4.

Step 1340, the target upgrade service module sends the target upgrade package to the target medical device.

It should be understood that although the individual steps in the flowcharts involved in the embodiments as described above are shown sequentially as indicated by the arrows, these steps are not necessarily executed sequentially in the order indicated by the arrows. Unless expressly stated herein, there is no strict order limitation on the execution of these steps, and these steps may be executed in other orders. Moreover, at least a portion of the steps in the flowchart involved in the embodiments as described above may comprise multiple steps or multiple phases, which are not necessarily executed at the same moment of completion, but may be executed at different moments, and the order of execution of these steps or phases is not necessarily sequential, but may be performed in turn or alternately with at least a portion of the other steps or steps in the other steps.

In some embodiments, the present disclosure also provides a computer device, the computer device comprises a memory and a processor, the memory stores a computer program, the processor implements methods of the present disclosure (e.g., the method 1200) when executing the computer program.

In some embodiments, the present disclosure also provides a storage medium, the storage medium stores a computer program, methods of the present disclosure (e.g., the method 1200) are performed when the computer program is executed by a processor.

The basic concepts have been described above, and it is apparent to those skilled in the art that the foregoing detailed disclosure is intended as an example only and does not constitute a limitation of the present disclosure. While not expressly stated herein, various modifications, improvements, and amendments may be made to the present disclosure by those skilled in the art. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure such as “an embodiment”, “one embodiment”, and/or “some embodiment”, which means a feature, structure, or characteristic associated with at least one embodiment of the present disclosure. Accordingly, it should be emphasized and noted that “one embodiment”, “an embodiment” or “an alternative embodiment” referred to two or more times in different locations in the present disclosure means a feature, structure, or characteristic related to at least one embodiment of the present disclosure. Therefore, it should be stressed and notes that “one embodiment” or “an alternative embodiment” in different places in the present disclosure do not necessarily refer to the same embodiment. In addition, certain features, structures, or characteristics in one or more embodiments of the present disclosure may be suitably combined.

In addition, unless expressly stated in the claims, the order of processing elements and sequences, the use of numerical letters, or the use of other names as described herein are not intended to qualify the order of the processes and methods of the present disclosure. While some embodiments of the invention that are currently considered useful are discussed in the foregoing disclosure by way of various examples, it should be appreciated that such details serve only illustrative purposes, and that additional claims are not limited to the disclosed embodiments, rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the disclosure of the present disclosure, and thereby aid in the understanding of one or more embodiments of the invention, the foregoing descriptions of embodiments of the present disclosure sometimes group multiple features together in a single embodiment, accompanying drawings, or a description thereof. However, this method of disclosure does not imply that the objects of the present disclosure require more features than those mentioned in the claims. Rather, claimed subject matter may lie in less than all features of a single foregoing disclosed embodiment.

Numbers describing the number of components, attributes, and attributes are used in some embodiments, and it should be understood that such numbers used in the description of embodiments are modified in some examples by the modifiers “about”, “approximately”, or “generally”. Unless otherwise noted, the terms “about”, “approximately”, or “generally” indicates that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments, such values are set to be as precise as possible within a feasible range.

For each patent, patent application, patent application disclosure, and other material cited in the present disclosure, such as articles, books, specification sheets, publications, documents, etc., the entire contents of which are hereby incorporated herein by reference. Historical application history documents that are inconsistent with or create a conflict with the contents of the present disclosure are excluded, as well as documents that limit the broadest scope of the claims of the present disclosure (currently or hereafter appended to the present disclosure). It should be noted that in the event of any inconsistency or conflict between the descriptions, definitions, and/or use of terminology in the materials appended to the present disclosure and those set forth in the present disclosure, the descriptions, definitions and/or use of terminology in the present disclosure prevail.

Finally, it should be understood that the embodiments described in the present disclosure are used only to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A system for upgrading medical devices, comprising a registration center and at least one upgrade server, and at least one medical device, the registration center being connected to the at least one upgrade server and the at least one medical device, and at least one upgrade service module being deployed on the at least one upgrade server, each of the at least one upgrade service module being deployed on one of the at least one upgrade server, wherein:
the registration center is configured for, in response to an upgrade request sent by a target medical device, determining a target upgrade service module from the at least one upgrade service module and sending registration information of the target upgrade service module to the target medical device;
the target medical device is configured for establishing a communication connection with the target upgrade service module based on the registration information and sending device information to the target upgrade service module through the communication connection; and
the target upgrade service module is configured for determining a target upgrade package matching the target medical device based on the device information and sending the target upgrade package to the target medical device.

2. The system of claim 1, wherein the determining a target upgrade service module from the at least one upgrade service module comprises:
determining the target upgrade service module from the at least one upgrade service module based on first load information of each upgrade service module.

3. The system of claim 2, wherein the determining the target upgrade service module from the at least one upgrade service module based on first load information of each upgrade service module comprises:
for each upgrade service module, obtaining the first load information of the upgrade service module and second load information of an upgrade server on which the upgrade service module is deployed; and
determining the target upgrade service module from the at least one upgrade service module based on the first load information and the second load information corresponding to each upgrade service module.

4. The system of claim 1, wherein when the registration center receives upgrade requests from a plurality of target medical devices at the same time, the registration center is further configured for:
determining a priority ranking of the plurality of target medical devices based on device information of the plurality of target medical devices; and
determining a corresponding target upgrade service module for each of the plurality of target medical devices in turn based on the priority ranking of the plurality of target medical devices.

5. The system of claim 1, wherein before receiving the upgrade request sent by the target medical device, the registration center is further configured for:
receiving registration information sent by the at least one upgrade service module and store the registration information.

6. The system of claim 1, wherein
one of the at least one upgrade service module is configured for sending a wake-up message to the target medical device based on a preset wake-up time; and
the target medical device is further configured for sending the upgrade request to the registration center in response to the wake-up message.

7. The system of claim 6, wherein:
when the target medical device is in a shutdown state or a standby state at a time of receiving the wake-up message, the sending the upgrade request to the registration center in response to the wake-up message comprises: powering up and sending the upgrade request to the registration center; or
when the target medical device is in a working state at the time of receiving the wake-up message, the sending the upgrade request to the registration center in response to the wake-up message comprises: sending the upgrade request to the registration center after the target medical device has finished a current workflow.

8. The system of claim 1, wherein the device information comprises version information of the target medical device, and the determining a target upgrade package matching the target medical device based on the device information received further comprises:
determining a current version of the target medical device based on the version information;
in response to determining that the current version is lower than a target version corresponding to the target upgrade service module, determining an upgrade link for upgrading to the target version from the current version; and
determining, based on the upgrade link, the target upgrade package matching the target medical device from at least one upgrade package of the target upgrade service module.

9. The system of claim 8, wherein the at least one upgrade package of the target upgrade service module is obtained from a remote management device through a gateway.

10. The system of claim 1, wherein the target upgrade service module is further configured for:
obtaining an upgrade result of the target medical device; and
sending the upgrade result to a remote management device through a gateway.

11. The system of claim 1, wherein the registration center is further configured for:
for each upgrade service module,
sending a heartbeat signal to the upgrade service module;
determining a connection state between the upgrade service module and the registration center based on a response of the upgrade service module to the heartbeat signal; and
in response to determining that the connection state is a disconnected state, deleting the registration information of the upgrade service module.

12. The system of claim 1, wherein the registration center and the at least one upgrade server are deployed on a same server.

13. A method for device upgrading, implemented by a target medical device, comprising:
sending an upgrade request to a registration center such that the registration center determines a target upgrade service module from at least one upgrade service module based on the upgrade request;
receiving registration information of the target upgrade service module sent by the registration center;
establishing a communication connection with the target upgrade service module based on the registration information and sending device information to the target upgrade service module through the communication connection such that the target upgrade service module determines a target upgrade package based on the device information; and
receiving the target upgrade package sent by the target upgrade service module and performing device upgrading based on the target upgrade package.

14. A method for device upgrading, implemented on a target upgrade service module deployed on an upgrade server, the upgrade server being connected to a registration center, the method comprising:
establishing a communication connection to a target medical device, wherein the target upgrade service module is determined from at least one upgrade service module deployed on the upgrade server by the registration center in response to an upgrade request sent by a target medical device;
receiving device information from the target medical device through the communication connection;
determining a target upgrade package matching the target medical device based on the device information; and
sending the target upgrade package to the target medical device.

15. The method of claim 14, wherein the target upgrade service module is determined from the at least one upgrade service module based on first load information of each upgrade service module.

16. The method of claim 15, wherein the target upgrade service module is determined from the at least one upgrade service module based on the first load information of each upgrade service module by:
for each upgrade service module, obtaining the first load information of the upgrade service module and second load information of an upgrade server on which the upgrade service module is deployed; and
determining the target upgrade service module from the at least one upgrade service module based on the first load information and the second load information corresponding to each upgrade service module.

17. The method of claim 14, wherein the device information comprises version information of the target medical device, and determining the target upgrade package matching the target medical device based on the device information further comprises:
determining a current version of the target medical device based on the version information;
in response to determining that the current version is lower than a target version corresponding to the target upgrade service module, determining an upgrade link for upgrading to the target version from the current version; and
determining, based on the upgrade link, the target upgrade package matching the target medical device from at least one upgrade package of the target upgrade service module.

18. The method of claim 17, wherein the at least one upgrade package of the target upgrade service module is obtained from a remote management device through a gateway.

19. The method of claim 14, further comprising:
obtaining an upgrade result of the target medical device; and
sending the upgrade result to a terminal device through a gateway.

20. The method of claim 14, wherein the registration center and the at least one upgrade server are deployed on a same server.
